(19) United States
(12) Patent Application Publication
(10) Pub. No.: US 2020/0214568 A1
(43) Pub. Date: Jul. 9, 2020

(54) MULTIMODAL IMAGING SYSTEM AND METHOD

(71) Applicant: CapeRay Medical (Pty) Ltd., Westlake (ZA)

(72) Inventors: Christopher Leonard Vaughan, Newlands (ZA); Roland Victor Baasch, Cape Town (ZA)

(21) Appl. No.: 16/647,809

(22) PCT Filed: Sep. 21, 2018
(86) PCT No.: PCT/IB2018/057291

§ 371 Date: Mar. 16, 2020

(30) Foreign Application Priority Data
Sep. 22, 2017 (GB) 1715412.1

(51) Int. Cl. A61B 5/00; A61B 6/00; A61B 6/02; A61B 8/08; A61B 8/00

(52) U.S. Cl. CPC A61B 5/0035; A61B 8/5261; A61B 5/0077; A61B 5/0091; A61B 6/4417; A61B 5/704; A61B 6/025; A61B 6/502; A61B 8/0825; A61B 8/4218; A61B 8/4416; A61B 6/4007; A61B 6/4464; A61B 6/5247; A61B 5/0064

(57) ABSTRACT

An imaging assembly, system and method for automated multimodal imaging of biological tissue is provided. It finds particular application, although in no way exclusively, in the medical imaging of breast tissue. An optical 3D scanner is included to determine the shape of the surface of both breasts and output a plurality of 3D coordinates thereof. An X-ray generator is included for sequentially radiating X-rays at a plurality of angles, through the tissue, toward an X-ray detector positioned below the patient and thus the breasts. An articulated arm holding an ultrasound transducer at an end thereof automatically moves the ultrasound transducer along a path defined by the obtained 3D coordinates for ultrasound imaging of the breasts while maintaining the transducer in contact with the surface at an orientation required for ultrasound imaging.

### CROSS-REFERENCE(S) TO RELATED APPLICATIONS

This application claims priority from United Kingdom patent application number 1715412.1 filed on 22 Sep. 2017, which is incorporated by reference herein.

### FIELD OF THE INVENTION

The invention disclosed herein relates to medical imaging. More particularly, it relates to multimodal medical imaging in which images obtained using two or more imaging modalities are geometrically co-registered to create resulting output imagery.

### BACKGROUND TO THE INVENTION

Multimodal imaging refers to the general principle of co-registering or integrating imagery obtained from two or more different imaging technologies. In doing so, the strengths of one or more of the imaging technologies used in a particular multimodal imaging technique may be utilised to supplement the resulting output imagery where deficiencies are presented by the remainder of the imaging technologies used in the particular technique. For example, a number of known multimodal imaging techniques utilise a combination of X-ray imaging and ultrasound imaging.

Certain known methods of imaging breast tissue, such as mammography, require the breast to be compressed between two compression plates for the imaging to have usable results. This is known to be particularly uncomfortable to the patient. This may present a barrier to voluntary preventative screening that may otherwise lead to early detection of abnormalities, since the discomfort associated therewith may be discouraging to the patient.

It has been established that the application of full-field digital mammography (FFDM), which is based on X-ray imaging, followed by automated breast ultrasound (ABUS), which is based on ultrasound imaging, leads to a more effective and accurate diagnosis of carcinoma or other abnormalities in breast tissue. Four general apparatus designs are known that include both FFDM and ABUS in the same apparatus.

In the first known design, X-ray images are captured by a flat panel digital detector located beneath the breast being examined and an ultrasound probe located above the breast. This probe, which is moved under automated control on top of the compressor plate, is positioned between the X-ray tube and the breast. Shortcomings of this design include that the two sets of images must be gathered sequentially, rather than simultaneously, thus increasing the breast compression time; the small size of the ultrasound probe means that multiple scans are required to cover the whole breast, further increasing compression time; and, because flat-panel detectors suffer from scatter problems, the radiation exposure to the patient is higher than optimal.

In a second known design, which is essentially an adaptation of the first design, digital breast tomosynthesis (DBT) is further incorporated into the apparatus enabling the construction of three-dimensional images of the breast. However, sequential image capture is still required with multiple scans by the ultrasound probe and may result in high radiation exposure to the patient.

A third known design is based on an FFDM system that uses a slot-scanning approach to acquire a planar X-ray image of the breast. Since this design utilizes an X-ray detector that moves beneath the breast platform, it allows the ultrasound probe to be located parallel to the X-ray detector. With the X-ray detector and the ultrasound probe both being located beneath the breast platform, it allows the acquisition of the X-ray and ultrasound images simultaneously, while the slot-scanning geometry reduces scatter and therefore minimizes radiation exposure to the patient. However, the X-ray fan beam in this design is created by a rotating X-ray tube, with the effect that the system cannot acquire three-dimensional images, but merely two-dimensional images. Furthermore, this design fails to adequately address the problem of acoustically coupling the ultrasound probe to the breast.

A fourth known design requires the patient to lie on her stomach in the prone position on a horizontal support with her breasts protruding through an opening in the support. Both the X-ray and ultrasound acquisition systems are located beneath the support and rotate around the breast, enabling the capture of three-dimensional images in both modalities. While this design has the advantage of not requiring compression of the breast, the breast tissue in the axilla or close to the chest wall cannot be imaged since the breast does not protrude far enough into the imaging field. Furthermore, the method of acquiring three-dimensional X-ray images, which is based on computed tomography, could expose the patient to an unnecessarily high radiation dose.

The applicant's own granted U.S. Pat. No. 9,636,073 discloses a scanning assembly for a dual-modality automated biological tissue imaging device having first and second compression surfaces. The assembly has a housing defining a scanning and compression surface. An ultrasound transducer is mounted within the housing adjacent the scanning surface for movement in a plane parallel to the scanning surface and for imaging the tissue through the scanning surface. An X-ray detector is also mounted within the housing for forming an X-ray image of the tissue based on X-ray radiation passed through the tissue and scanning surface from an X-ray source. It further includes a drive for moving the transducer across the scanning surface so that the transducer generates a plurality of two-dimensional ultrasound tissue images. The housing is hermetically sealed and filled with non-conductive fluid with acoustic impedance resembling that of the tissue. The scanning surface has acoustic impedance resembling that of the tissue, thereby addressing acoustic coupling of the ultrasound probe to the breast, and can substantially withstand compression forces applied to the tissue.

The applicant considers there to be scope for further improvement through the invention disclosed herein, which also addresses at least some of the issues mentioned above, at least to some extent.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with this disclosure there is provided an imaging assembly for an automated multimodal imaging system for imaging biological tissue, comprising:
a support for supporting the tissue during imaging;
an optical 3D scanner for obtaining a plurality of 3D coordinates of a surface of the tissue;
an X-ray generator for sequentially radiating X-rays at a plurality of angles, through the tissue, toward an X-ray detector positioned below the tissue; and
an articulated arm arranged to hold an ultrasound transducer at an end thereof and to automatically move the ultrasound transducer along a path defined by the obtained 3D coordinates for ultrasound imaging of the tissue while maintaining the transducer in contact with the surface at a predetermined orientation.

The 3D scanner may include a camera positioned above the support and arranged to be moved relative to the support to sequentially position the camera at a plurality of angles relative to the support. Alternatively, the 3D scanner may include a plurality of cameras positioned above the support and spaced apart from one another at a plurality of angles relative to the support.

The X-ray generator may be arranged to be moved relative to the support to sequentially position the X-ray generator at a plurality of angles relative to the support. Alternatively, the assembly may include a plurality of X-ray generators that are spaced apart from one another at a plurality of angles relative to the support.

The X-ray detector may be integrally formed with the support. Alternatively, the X-ray detector may be positioned below the support. The X-ray detector may be stationary or movable in a plane parallel to the support. In one embodiment, the X-ray detector is a flat panel X-ray detector.

The arm and/or transducer may include a pressure sensor to measure a pressure between the transducer and the surface of the tissue when in contact therewith. The predetermined orientation of the transducer may be in a direction substantially orthogonal to the surface of the tissue.

In a second aspect of the disclosure there is provided an automated multimodal imaging system for imaging biological tissue, the system including an imaging computing device comprising:
a 3D coordinate obtaining module for receiving optical data from an optical 3D scanner and obtaining a plurality of 3D coordinates of a surface of the tissue from the received data;
an X-ray generator module configured to sequentially cause an X-ray generator to radiate X-rays at a plurality of angles, through the tissue, toward an X-ray detector positioned below the tissue;
an X-ray detector module for converting a sequence of X-rays received by the X-ray detector into X-ray data; and
an articulated arm module for automatically moving an articulated arm with an ultrasound transducer at an end thereof along a path defined by the obtained 3D coordinates, and for maintaining the transducer in contact with the surface of the tissue at an orientation for imaging of the tissue in a direction substantially orthogonal to the surface; and
an ultrasound module for obtaining ultrasound data from the ultrasound transducer.

The imaging computing device may include an X-ray image module arranged to generate a plurality of two-dimensional X-ray images of the tissue from the X-ray data. The imaging computing device may include an ultrasound image module for generating a plurality of two-dimensional ultrasound images of the tissue from the ultrasound data.

The 3D coordinate obtaining module may furthermore be arranged to receive a plurality of digital images from one or more cameras, the digital images captured at a plurality of angles relative to the tissue. The 3D coordinate obtaining module may configure an angle of the one or more cameras relative to the tissue.

The X-ray generator controller may furthermore be arranged to configure an angle of the X-ray generator relative to the tissue. The X-ray detector module may furthermore be arranged to move the X-ray detector in a substantially horizontal plane.

The imaging computing device may further include a volumetric reconstruction module for generating, from the X-ray images, a first volumetric reconstruction of the tissue and for generating, from the ultrasound images, a second volumetric reconstruction of the tissue. The imaging computing device may include an image co-registering module for co-registering the first and second volumetric reconstructions to generate a third volumetric reconstruction of the tissue.

The computing device may include a slice reconstruction module for reconstructing image slices of one or more of the volumetric reconstructions. The slice reconstruction module may make use of tomosynthesis algorithms to reconstruct the image slices.

In a third aspect of the disclosure there is provided a method for automatically imaging biological tissue positioned on a support, the method comprising
obtaining a plurality of 3D coordinates of a surface of the tissue by means of a 3D scanner;
causing an X-ray generator to sequentially radiate X-rays at a plurality of angles, through the tissue, toward an X-ray detector positioned below the tissue;
converting a sequence of X-rays received by the X-ray detector into X-ray data; and
automatically moving an articulated arm having an ultrasound transducer at an end thereof along a path defined by the obtained 3D coordinates while maintaining the transducer in contact with the surface of the tissue at a predetermined orientation, and obtaining ultrasound data from the ultrasound transducer.

The method may further include: generating a plurality of two-dimensional X-ray images of the tissue from the X-ray data. The method may further include generating a plurality of two-dimensional ultrasound images of the tissue from the ultrasound data.

The biological tissue may be breast tissue of a patient. The step of providing the tissue to be imaged on the support may include: donning the patient with a tight-fitting camisole made from material that is rendered acoustically transparent when impregnated with ultrasound gel; impregnating the camisole over the breast tissue with ultrasound gel; and positioning a patient in a supine position on the support.

The 3D coordinates may include 3D coordinates of both breasts of the patient.

The step of causing an X-ray generator to sequentially radiate X-rays at a plurality of angles through the tissue may include: moving the X-ray detector into a first position, determined from the 3D coordinates, for detecting X-rays radiated through a first breast of the patient; causing the X-ray generator to sequentially radiate X-rays at a plurality of angles, through the first breast, toward the X-ray detector positioned below the first breast; moving the X-ray detector into a second position, determined from the 3D coordinates, for detecting X-rays radiated through a second breast of the patient; and causing the X-ray generator to sequentially radiate X-rays at a plurality of angles, through the second breast, toward the X-ray detector positioned below the second breast.

The step of automatically moving the articulated arm and the ultrasound transducer along the path defined by the obtained 3D coordinates may be performed for both breasts using the same set of obtained 3D coordinates, such that the step of generating a plurality of two-dimensional ultrasound images of the tissue generates a plurality of two-dimensional images for both breasts of the patient.

The two-dimensional X-ray images may be X-ray images in a coronal plane. The ultrasound images may be ultrasound images in a sagittal plane.

The method may further include the steps of: generating, from the X-ray images, a first volumetric reconstruction of the tissue; generating, from the ultrasound images, a second volumetric reconstruction of the tissue; and co-registering the first and second volumetric reconstructions to generate a third volumetric reconstruction of the tissue.

In each aspect of the disclosure, a particular feature or step described may form part of an embodiment alone or in combination with any one or more features or steps.

An embodiment of the disclosure will now be described, by way of example only, with reference to the accompanying drawings.

### BRIEF DESCRIPTION OF THE DRAWINGS

In the drawings:

FIG. 1 is a schematic representation of part of an imaging assembly;

FIG. 2A is a schematic representation of a further part of the imaging assembly of FIG. 1;

FIG. 2B to 2G are schematic representations of the assembly of FIG. 2A showing X-rays radiated at different angles;

FIG. 3A to 3F are schematic representations of the assembly of FIG. 2A in a further X-ray detecting configuration;

FIG. 4A is a schematic representation of a further part of the imaging assembly of FIG. 1 including an articulated arm carrying an ultrasound probe;

FIG. 4B to 4G are schematic representations of the assembly of FIG. 4A showing the articulated arm at various points along a surface path of a left breast of a patient during ultrasound imaging;

FIG. 5A to 4F are schematic representations of the assembly of FIG. 4A showing the articulated arm at various points along a surface path of a right breast of a patient during ultrasound imaging;

FIG. 6A is a schematic representation of the X-ray components of an alternative exemplary imaging assembly;

FIG. 6B is a schematic representation of an X-ray exposure area on the X-ray detector shown in FIG. 6A;

FIG. 7 is a block diagram of an image computing device useful in a system and method according to the disclosure;

FIG. 8 is a flow diagram of a method for automatically imaging biological tissue according to the disclosure; and

FIG. 9 illustrates an example of a computing device in which various aspects of the disclosure may be implemented.

### DETAILED DESCRIPTION WITH REFERENCE TO THE DRAWINGS

An imaging assembly for an automated multimodal imaging system is provided for imaging biological tissue. Furthermore, an imaging system configured to control the imaging assembly and a method of operating the assembly are also provided. It finds particular application, although in no way exclusively, in the in vivo medical imaging of breast tissue. This exemplary application will be used throughout the description unless the context indicates otherwise.

In the application of breast imaging, the method does not require the breast to be mechanically compressed and the patient is therefore not exposed to the normal discomfort otherwise associated with compression techniques. Instead, the patient lies in a supine position on a support, for example a gurney, and the breasts are naturally compressed under natural gravitational force. An optical 3D scanner is included to remotely determine the shape of the surface of both breasts and output a plurality of 3D coordinates thereof. An X-ray generator is included for sequentially radiating X-rays at a plurality of angles, through the tissue, toward an X-ray detector positioned below the patient and thus the breasts. An articulated arm holding an ultrasound transducer at an end thereof automatically moves the ultrasound transducer along a path defined by the obtained 3D coordinates for ultrasound imaging of the breasts while maintaining the transducer in contact with the surface at an orientation required for ultrasound imaging.

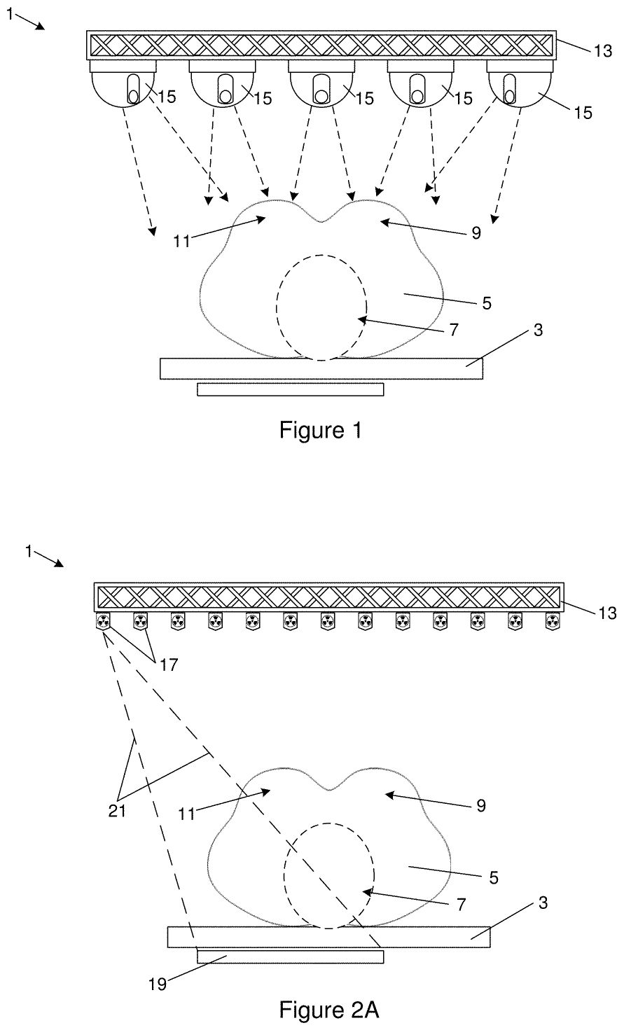

FIG. 1 shows a schematic representation of part of an imaging assembly (1) for an automated multimodal imaging system according to the disclosure. The assembly (1) includes a support, presently a gurney (3) on which the patient (5) is supported, lying in the supine position. FIG. 1 shows the patient (5) lying in the supine position on the gurney (3) as seen from the patient's head (7). In this position, the breasts (9, 11) of the patient are naturally compressed under the force of gravity. Overhead and spaced apart from the gurney (3), a gantry (13) is provided on which various components of the assembly (1) are provided as will be described in further detail below.

Also shown in FIG. 1, are a plurality of cameras (15) spaced apart from one another on the gantry (13) and orientated at different angles relative to the gurney (3) (and thus the patient (5) during use). In the present embodiment, the cameras (15) form part of an optical 3D scanner for remotely determining the shape of the surface of both breasts (9, 11). During use, the cameras (15) capture images of the breasts (9, 11) at different angles. This allows an imaging computing device controlling the assembly (1) to process the captured images using known 3D shape acquisition techniques and determine a plurality of 3D coordinates that each correspond to a point on the surface of the breasts (9, 11). One such exemplary 3D shape acquisition technique is disclosed in Zhang S, “Recent progresses on real-time 3D shape measurement using digital fringe projection techniques”, Optics and Lasers in Engineering, 48(2): 149-158, 2010.

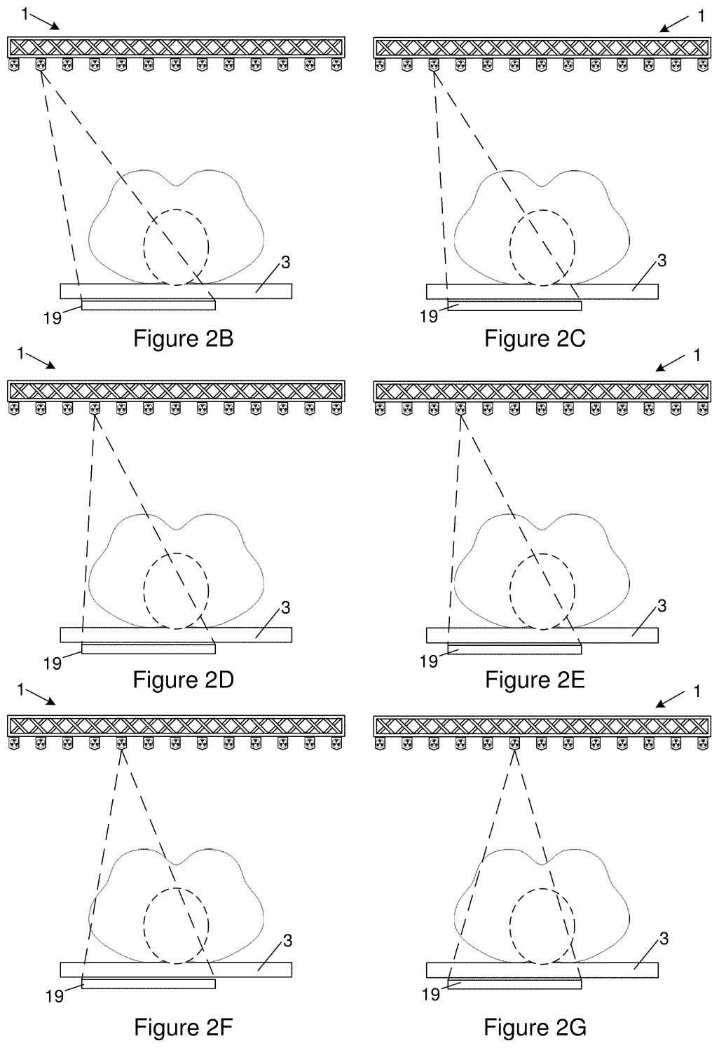

FIG. 2A shows another part of the assembly (1) in which like reference numerals indicates like features to those of FIG. 1. The cameras (15) shown in FIG. 1 have been omitted in FIG. 2A to more clearly show further components of the assembly (1). The assembly further includes a series of stationary X-ray sources or generators (17) provided on, and spaced apart on the gantry (13). The assembly (1) further includes a flat panel X-ray detector (19) positioned below the gurney (3) that is movable in a plane substantially parallel to the gurney (3) to position the flat panel X-ray detector (19) for X-ray imaging in a desired plane through the patient (5).

FIG. 2A shows the assembly (1) in a configuration for X-ray imaging of the left breast (11) of the patient (5). Having obtained the 3D coordinates of the left breast (11), the X-ray detector may be positioned at an appropriate location below the patient (5) required for detecting X-rays radiated through the left breast (11). The X-ray generators (17) that are located in the relevant positions to image a particular breast, presently the left breast (11), may then be sequentially energised to emit X-rays at different angles relative to the patient (5). This causes X-rays to radiate at different incident angles through the breast (11) and onto the X-ray detector (19). FIG. 2A shows a first X-ray generator (17) radiating X-rays (21) in a first angle relative to the patient (5), onto the flat panel X-ray detector (19).

FIGS. 2B through 2G show subsequent X-ray generators (17) sequentially radiating X-rays at different angles, thereby enabling a plurality of coronal plane slices of the left breast (11) to be reconstructed from data generated by the X-ray detector (19) in response to detecting each respective X-ray emission from the particular X-ray generator (17).

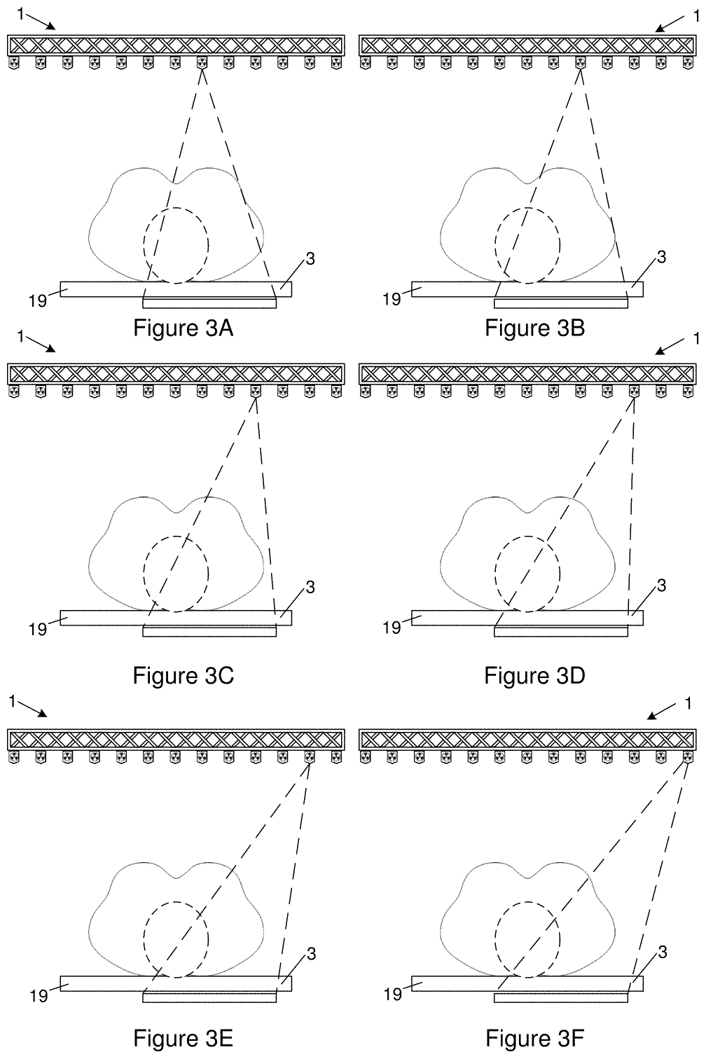

Once X-ray imaging of the left breast (11) is complete, the flat panel X-ray detector (19) may be moved and positioned at an appropriate location below the patient (5) required for detecting X-rays radiated through the right breast (9), as shown in FIG. 3A. FIGS. 3A through 3F show the X-ray generators (17) in the relevant orientations to image the right breast (9), with generators (17) radiating X-rays at various angles through the right breast (9) and onto the X-ray detector (19). This enables a plurality of coronal plane slices of the right breast (9) to be reconstructed from data generated by the X-ray detector (19), as described above with reference to the left breast (11).

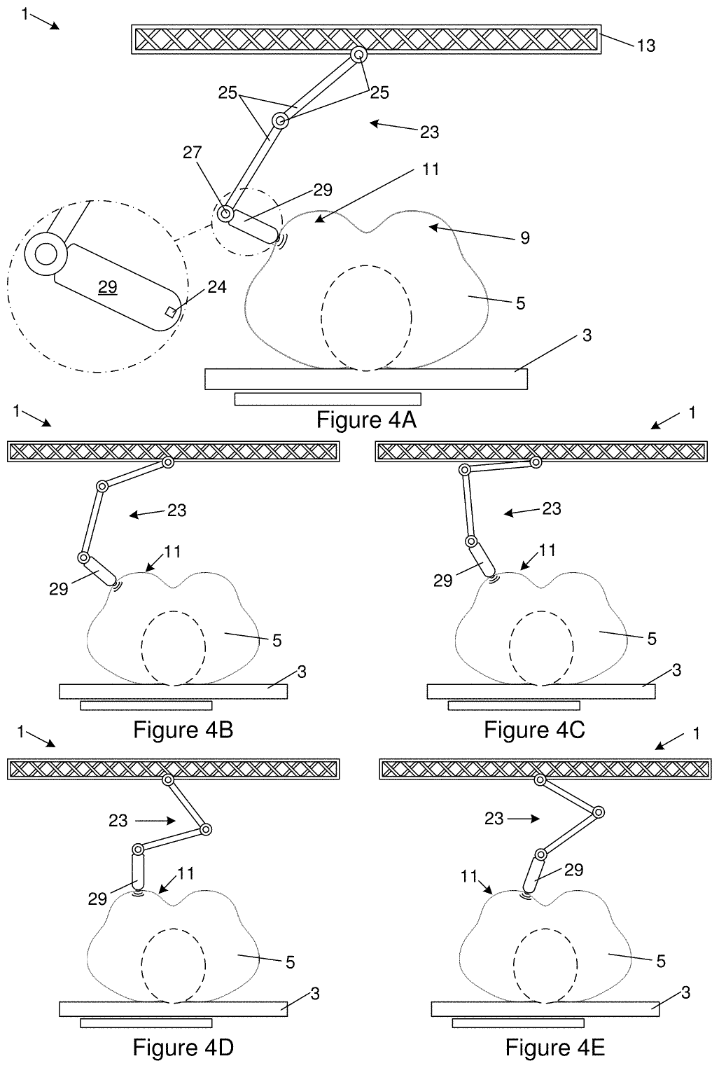

FIG. 4A shows the assembly (1) further including an articulated arm (23) attached to the gantry (13). The arm (23) has two segments (25) connected by joints (27) allowing the segments to move relative to one another. The arm (23) includes actuators (not shown) configured to move the segments of the arm in response to a control signal received thereby. The arm (23) has an ultrasound transducer (29) held at a free end thereof. The arm (23) is operable to move the ultrasound transducer (29) to a position within its reach and at a desired orientation through manipulation of the joints (27).

The arm (23) is operable to automatically move the ultrasound transducer (29) along a path defined by the obtained 3D coordinates for ultrasound imaging of the breasts (9, 11). The arm (23) is further operable to maintain the ultrasound transducer (29) in contact with the surface of the relevant breast (9, 11) at a predetermined orientation. This predetermined orientation may be required to ensure optimal ultrasound imaging of the tissue. In the present embodiment, imaging substantially orthogonal to the surface of the tissue being imaged may be preferable. The ultrasound transducer (29) further includes a pressure sensor (24) at its tip configured to measure pressure between the ultrasound transducer (29) and the surface of the breast (11). This provides an imaging computing device, controlling the assembly (1), with feedback to enable it to maintain adequate pressure between the ultrasound transducer (29) and the breast (11) as may be required by the particular ultrasound transducer for proper imaging. The pressure feedback may further provide a safety mechanism to ensure that the patient does not experience undue discomfort due to possible excessive force being applied by the ultrasound transducer.

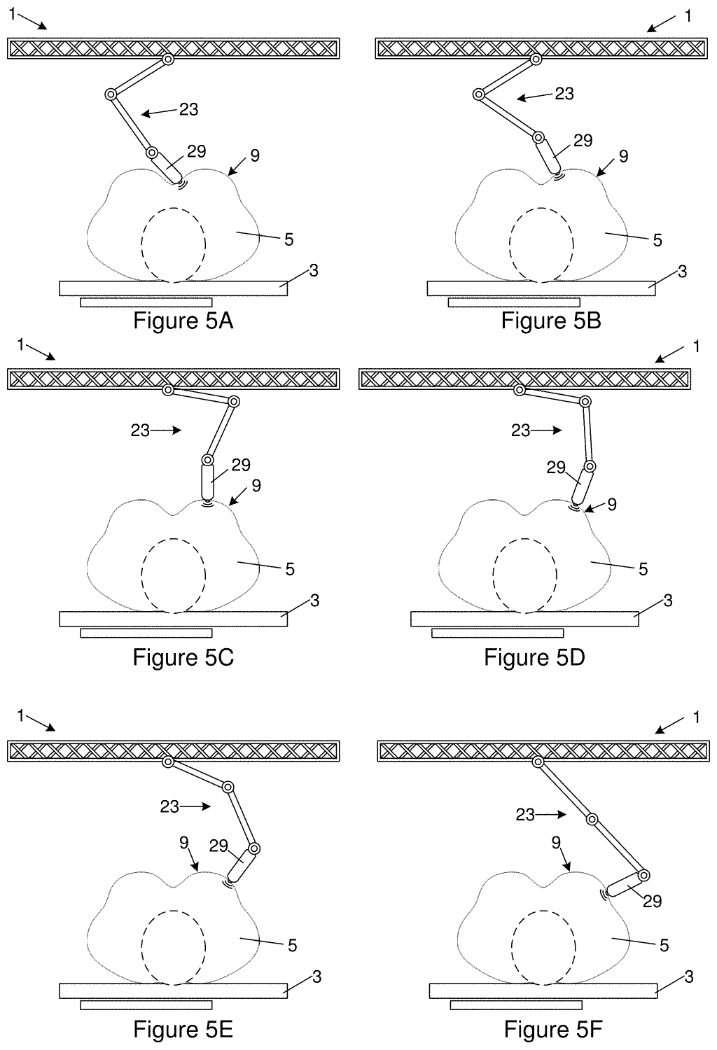

FIG. 4A shows the arm (23) holding the ultrasound transducer (29) in a first position for imaging of the left breast (11) of a patient, with the ultrasound transducer (29) held substantially orthogonal to the surface of the breast (11). As shown in FIGS. 4A through 4E, the arm (23) is operable to automatically move the ultrasound transducer (29) along surface of the left breast (11) as defined by the obtained 3D coordinates thereof. This allows the obtaining of ultrasound imaging of the entire breast. The procedure may be repeated for the right breast (9) as shown in FIGS. 5A through 5F. In this manner, both breasts (9, 11) may be automatically imaged, without operator intervention, using the ultrasound transducer (29), thereby producing ultrasound imaging data for both breasts.

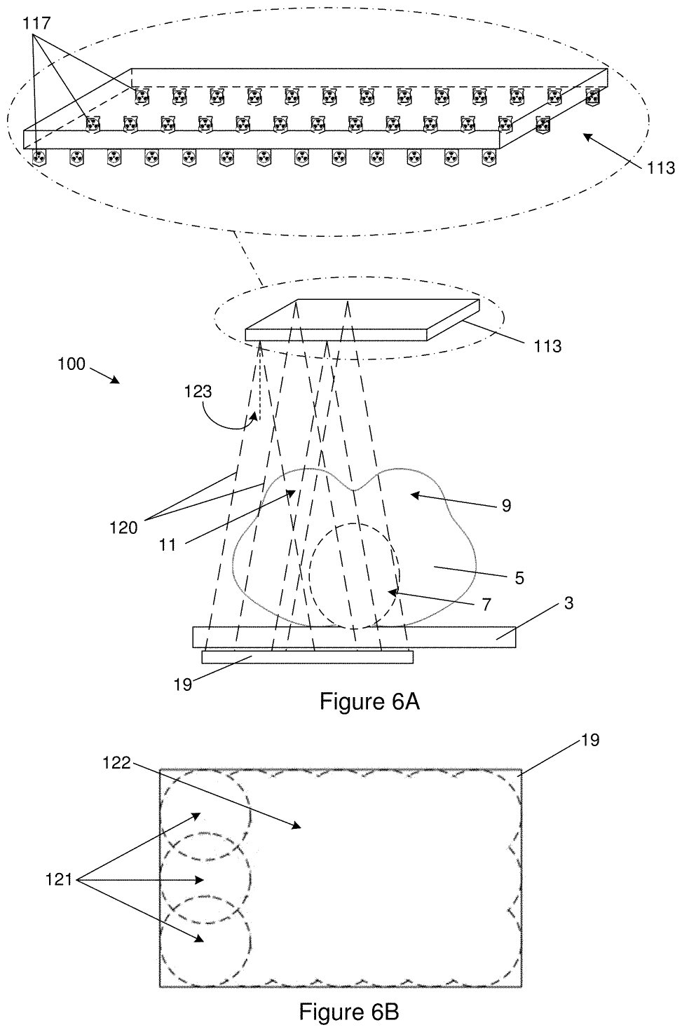

FIG. 6A shows an alternative embodiment of the assembly (100) that differs in respect to the assembly (1) described above in that it has an X-ray generator (113) with a number of X-ray emitters (117) arranged in rows and columns to form a grid of X-ray emitters (117). One such exemplary X-ray generator (113) is disclosed in PCT publication number WO2017/130013 by Adaptix Ltd (“Adaptix”). It discloses a system that includes a flat panel array X-ray generator having a fixed two-dimensional array of X-ray emitters consisting of moderate pitch spacing in the mm to cm range between X-ray emitters.

The X-ray generator (113) shown in FIG. 6A has a 3×12 grid of X-ray emitters (117) and can be seen more clearly in the enlargement of the X-ray generator (113) in the ellipse of FIG. 6A. The number of X-ray emitters and the grid spacing is for exemplary purposes only and any number of rows and columns and/or total number of emitters may be used. Each X-ray emitter (117) is configured to radiate a substantially cone-shaped X-ray beam (120) toward the X-ray detector (19). In the present embodiment, the nominal angle (123) of the cone-shaped beam is 20° from the vertical.

As shown in FIG. 6B, the X-ray beam (120) irradiates a substantially circular area (121) if the irradiated object is planar and positioned normally to the beam, as the flat-panel X-ray detector (19) is in the present embodiment. The flat-panel X-ray generator (113), and thus the grid of X-ray emitters (117) are arranged such that the substantially circular exposure area (121) caused by each X-ray emitter (117) overlaps with that of adjacent X-ray emitters. The X-ray emitters (117) may be energised in quick succession to create an exposure area (122) that substantially irradiates most of the active area of the X-ray detector (19). Any of the normally substantially cone-shaped beams may be shaped by any means known in the art to obtain a desired resulting exposure area (122).

The flat-panel X-ray generator (113) may provide a number of advantages. The patient (5) may be subjected to a lower radiation burden than with known X-ray generators and the X-ray generator (113) may be positioned closer to the patient (5) and thus the breasts (9, 11). Further advantages include that the X-ray generator (113) remains stationary and therefore the entire assembly (100) may include less moving parts, which may make it less susceptible to mechanical failure and thus cheaper to maintain.

FIG. 6A shows the left breast (11) of the patient (5) being X-ray imaged using the X-ray generator (113) of the assembly (100). Having obtained the 3D coordinates of the left breast (11) in preceding steps, the X-ray detector (19) may be positioned at an appropriate location below the patient (5) required for detecting X-rays radiated through the left breast (11). The X-ray emitters (117) that are arranged in a grid and located in the X-ray generator (113) above the patient (5). The X-ray emitters (117) are all therefore positioned at different locations, i.e. different angles, relative to the patient (5) and thus the left breast (11) being imaged. The X-ray emitters (117) of the X-ray generator (113) may be sequentially energised to emit X-rays at different angles relative to the patient (5). This causes X-rays to radiate at different incident angles through the breast (11) and onto the X-ray detector (19). FIG. 6A shows four X-ray emitters (117) radiating X-rays (120) at various angles relative to the patient (5), onto the flat panel X-ray detector (19), however it should be understood that the X-ray emitters (117) may be energised sequentially and not simultaneously.

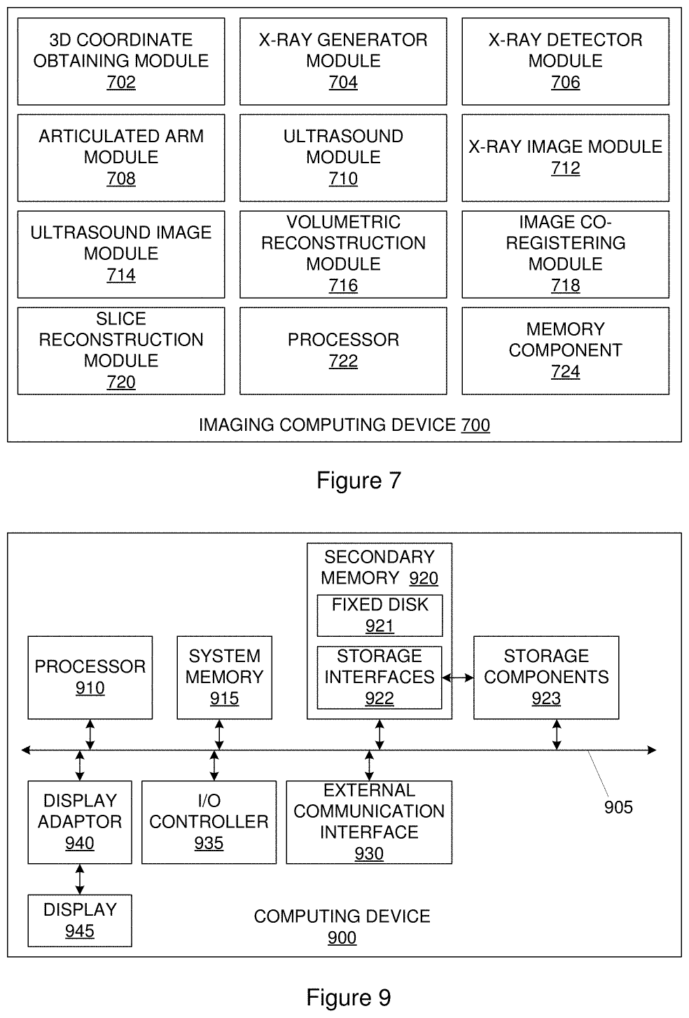

An imaging computing device (700) is shown in the block diagram of FIG. 7. The imaging computing device (700) may be configured to interface with and optionally control the imaging assembly (1) and its various sub-components. The imaging computing device includes a 3D coordinate obtaining module (702) that is configured to interface with the optical 3D scanner, in the above example including the cameras (15). More specifically, the 3D coordinate obtaining module (702) may be configured to control the cameras (15) individually so as to sequentially or simultaneously capture images from each camera (15) and to receive the optical data generated by each camera (15). The 3D coordinate obtaining module (702) is furthermore configured to process the received optical data to obtain a plurality of 3D coordinates of the surface of the tissue being imaged, in the present example both breasts (9, 11) of the patient (5). In the present embodiment, the 3D coordinate obtaining module (702) is configured to use image processing algorithms to determine the shape of the breasts (9, 11) and subsequently a plurality of 3D coordinates, each coordinate representing a point in three-dimensional space relative to a preconfigured reference point, in the present example the point of attachment of the articulated arm (23) to the gantry (13).

The imaging computing device (700) furthermore includes an X-ray generator module (704) configured to interface with and control the X-ray generators (17). The X-ray generator module (704) is, more particularly, configured to sequentially energise, or send control signals based on which energising is initiated, each X-ray generator (17). This causes each respective X-ray generator (17) to radiate X-rays at a respective preconfigured angle through the breast tissue, toward the X-ray detector (19). The X-ray generator module (704) is further arranged to configure the preconfigured radiating plane of each respective X-ray generator (17).

Further provided in the imaging computing device (700) is an X-ray detector module (706) configured to interface with and control the flat panel X-ray detector (19). The X-ray detector module (706) is also arranged to send control signals based on which the X-ray detector (19) moves in a substantially horizontal plane below the gurney (3) to position the X-ray detector (19) in an appropriate position for capturing X-rays radiated at a particular range of angles. For this purpose the X-ray detector may be mounted on one or more rails and may manually operable or electronically operable if fitted with a suitable drive motor. The X-ray detector module (706) is further configured to receive X-ray data from the X-ray detector (19) in response to the X-ray detector detecting the incidence of X-rays from the X-ray generators (17).

The imaging computing device (700) further includes an articulated arm module (708) for automatically moving the articulated arm (23) and ultrasound transducer (29) at its end along a path defined by the obtained 3D coordinates. The articulated arm module (708) is therefore in data communication with the 3D coordinate obtaining module (702) from which it receives the 3D coordinates obtained by the 3D coordinate obtaining module (702). The articulated arm module (708) calculates a path from the 3D coordinates and sends control signals to actuators controlling the movement of the arm segments (27) to sequentially move the tip of the ultrasound transducer (29) from one 3D coordinate to the next along the calculated path. The articulated arm module (708) is further configured to send control signals to the actuator at the end of the articulated arm (23) to maintain the ultrasound transducer (29) at a predetermined orientation relative to the path. In the present embodiment, the ultrasound transducer (29) requires a substantially orthogonal orientation relative to the surface of the tissue being imaged. The articulated arm module (708) is furthermore in data communication with the pressure sensor (24) to measure the pressure being applied by the ultrasound transducer (29) on the surface of the breast (9, 11). The articulated arm module (708) is further configured to send control signals to the actuators controlling the articulated arm (23) to maintain contact between the ultrasound transducer (29) and the breast (9, 11) at a preconfigured pressure.

The imaging computing device (700) further includes an ultrasound module (710) that is configured to interface with and control the ultrasound transducer (29) and to receive ultrasound data therefrom.

An X-ray image module (712) and an ultrasound image module (714) are provided that are in data communication with the X-ray detector module (706) and the ultrasound module (712) respectively. The X-ray image module (712) is configured to receive X-ray data obtained by the X-ray detector module (706) and to generate a plurality of two-dimensional X-ray images of the breasts from the X-ray data. Similarly, the ultrasound image module (714) is configured to receive ultrasound data obtained by the ultrasound module (710) and to generate a plurality of two-dimensional ultrasound images of the breasts from the ultrasound data.

The imaging computing device (700) includes a volumetric reconstruction module (716) that is in data communication with the X-ray image module (712) and the ultrasound image module (714) and configured to receive a plurality of two-dimensional X-ray images and a plurality of two-dimensional ultrasound images, or data representing both, from the respective modules (612, 614). The volumetric reconstruction module (716) is further configured to, from the X-ray images, generate a first volumetric reconstruction of the breasts and to, from the ultrasound images, generate a second volumetric reconstruction of the breasts.

The imaging computing device (700) further includes an image co-registering module (718) that is in data communication with the volumetric reconstruction module (716) and configured to receive the first and second volumetric reconstructions of the breasts therefrom. The image co-registering module (718) is further configured to geometrically co-register the first and second volumetric reconstructions to generate a third, combined, volumetric reconstruction of the breasts (9, 11). This may include transforming the first and second volumetric reconstructions of the breasts to have a common coordinate system to allow the image co-registering module (718) to integrate the different volumetric reconstructions.

The imaging computing device (700) may further include a slice reconstruction module (720) that is arranged to recreate image slices of the combined volumetric reconstruction of the breasts. The slice reconstruction component (720) may use tomosynthesis algorithms for the reconstruction of the image slices, generally for viewing by a technician or medical practitioner.

The imaging computing device (700) may include a processor (722) for executing the functions of modules described above, which may be provided by hardware or by software units executing on the imaging computing device. The software units may be stored in a memory component (724) and instructions may be provided to the processor (722) to carry out the functionality of the described components. In some cases, for example in a cloud computing implementation, software units arranged to manage and/or process data on behalf of the imaging computing device (700) may be provided remotely.

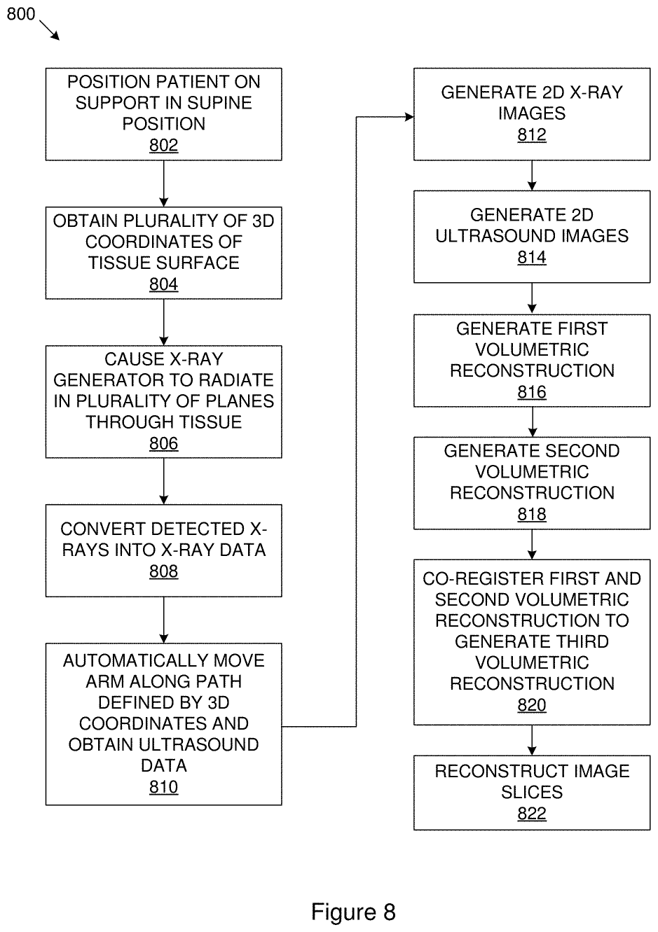

FIG. 8 shows a flow diagram of a method (800) for automatically imaging biological tissue, in the present example breast tissue of a patient. In a first step, the patient is positioned (802) on a support, the gurney (3) in the above example, in the supine position. It should be appreciated that the patient is also, before being so positioned, donned with a tight-fitting camisole that is rendered acoustically transparent by impregnating the camisole material with ultrasound gel. The 3D coordinate obtaining module (702) of the imaging computing device (700) then obtains (804) a plurality of 3D coordinates of a surface of the tissue being imaged, presently the breasts (9, 11) of the patient (5), by means of the 3D scanner of the imaging assembly (1), in the above example an array of cameras (13).

The X-ray generator module (704) then causes (806) the X-ray generators (17) to sequentially radiate X-rays at a plurality of angles, through the breasts (9, 11), toward the X-ray detector (19) positioned below the breasts. The X-ray detector module (706) then converts (808) a sequence of X-rays received by the X-ray detector (19) into X-ray data.

The articulated arm module (708) then automatically moves (810) the articulated arm (23) having the ultrasound transducer (29) at its end along a path defined by the obtained 3D coordinates, while maintaining the ultrasound transducer (29) in contact with the surface of the breasts (9, 11) at a predetermined orientation, and obtains ultrasound data from the ultrasound transducer.

The X-ray image module (712) then generates (812) a plurality of two-dimensional X-ray images of the breasts (9, 11) from the X-ray data; and the ultrasound image module (714) generates (814) a plurality of two-dimensional ultrasound images of the breasts (9, 11) from the ultrasound data.

The volumetric reconstruction module (716) then generates (816), from the X-ray images, a first volumetric reconstruction of the breasts (9, 11) and generates (818), from the ultrasound images, a second volumetric reconstruction of the breasts (9, 11). The image co-registering module (718) then co-registers (820) the first and second volumetric reconstructions to generate a third volumetric reconstruction of the breasts (9, 11).

After the volumetric reconstructions have been co-registered, the slice reconstruction module (720) may reconstruct (822) a number of image slices of the third and co-registered volumetric reconstruction by means of tomosynthesis algorithms. This may subsequently be presented to a technician or medical practitioner for viewing and diagnostic purposes.

Throughout the method (800), the patient (5) remains in substantially the same position. This is particularly required after the 3D coordinates of the surface of the breasts are obtained, since the remainder of the method relies on the accuracy of the obtained 3D coordinates.

Although the exemplary embodiments of the various aspects of the present disclosure have been described in terms of the imaging of breast tissue, it will be understood that the disclosure may be utilised for the automated multimodal imaging of any suitable biological tissue.

Furthermore, the 3D scanner need not comprise a plurality of cameras at various different angles relative to the tissue, but may comprise a single camera that is configured to be moved so that it may capture images of the tissue at various angles. Furthermore, the 3D scanner may comprise any suitable non-contact 3D scanning technology, such as a laser based time-of-flight 3D scanner, to name but one exemplary 3D scanning technology.

Similarly, the X-ray generator may be a single X-ray generator configured to be moved at different angles relative to the tissue, enabling it to radiate X-rays at various angles through the tissue. Furthermore, the X-ray detector may be integrally formed with the support.

While the exemplary method described above showed a single pass over the tissue of the breasts, it should be understood that multiple passes may be made across different transverse positions.

The present disclosure therefore provides a system and method for automatic multimodal imaging of biological tissue and, particularly, breast tissue. It is anticipated that the system and method described may cause little or no discomfort to the patient and radiation exposure to the patient may be limited since extended periods of X-ray exposure may be avoided. The system and method of the disclosure may further allow breast tissue in the axilla or located close to the chest wall to be imaged, allowing imaging of areas that may otherwise have been omitted.

A further advantage of the disclosed system and method is that, seeing as the patient's entire chest cavity or thorax is exposed to radiation, the system may be adapted to provide additional potentially useful images of the patient's lungs, heart and general thoracic cavity. It is envisaged that such adaptation may be done by simply adapting the software and other computational algorithms employed to reconstruct the images of the relevant organs or tissue.

A still further advantage of the system and method disclosed above is that the breast (or other tissue) of the patient is exposed to the same amount of compression (being gravitational compression) during both the X-ray and ultrasound scans. This has marked advantages in image reconstruction and co-registration.

The employment of the system and method as disclosed and in particular that the X-ray detector may be moved between the left and right breasts, depending on which is being scanned, also enables a smaller X-ray detector to be used. This is made possible because, unlike prior art scanning techniques, the scanning is done from side-to-side while the X-ray detector is moved in a horizontal plane to be beneath the breast being scanned. Smaller detectors may imply higher pixel density. Another advantage of the system and method described is that the X-ray detector may be held entirely stationary while a particular breast is being scanned. This is generally not possible in prior art scanners that scan the thorax from top-to-bottom or bottom-to-top, as opposed to side-to-side. It is foreseen that a flat panel X-ray detector of 228×291 mm having a pixel resolution of 4608×5888 and a pixel size of 49.5 microns may be used to conduct X-ray scanning in the embodiment disclosed.

It is also foreseen that the ultrasound probe used in the system and method of the invention may have a length of 192 mm with 768 detector elements at a pitch of 0.25 mm and operating at a centre frequency of 7 MHz. The scanning surface of the ultrasound probe may also be concaved to accommodate the shape of the breast and to facilitate increased contact between the probe and the breast surface.

FIG. 9 illustrates an example of a computing device (900) in which various aspects of the disclosure may be implemented. The computing device (900) may be embodied as any form of data processing device including a personal computing device (e.g. laptop or desktop computer), a server computer (which may be self-contained, physically distributed over a number of locations), a client computer, or a communication device, such as a mobile phone (e.g. cellular telephone), satellite phone, tablet computer, personal digital assistant or the like. Different embodiments of the computing device may dictate the inclusion or exclusion of various components or subsystems described below.

The computing device (900) may be suitable for storing and executing computer program code. The various participants and elements in the previously described system diagrams may use any suitable number of subsystems or components of the computing device (900) to facilitate the functions described herein. The computing device (900) may include subsystems or components interconnected via a communication infrastructure (905) (for example, a communications bus, a network, etc.). The computing device (900) may include one or more processors (910) and at least one memory component in the form of computer-readable media. The one or more processors (910) may include one or more of: CPUs, graphical processing units (CPUs), microprocessors, field programmable gate arrays (FPGAs), application specific integrated circuits (ASICs) and the like. In some configurations, a number of processors may be provided and may be arranged to carry out calculations simultaneously. In some implementations various subsystems or components of the computing device (900) may be distributed over a number of physical locations (e.g. in a distributed, cluster or cloud-based computing configuration) and appropriate software units may be arranged to manage and/or process data on behalf of remote devices.

The memory components may include system memory (915), which may include read only memory (ROM) and random access memory (RAM). A basic input/output system (BIOS) may be stored in ROM. System software may be stored in the system memory (915) including operating system software. The memory components may also include secondary memory (920). The secondary memory (920) may include a fixed disk (921), such as a hard disk drive, and, optionally, one or more storage interfaces (922) for interfacing with storage components (923), such as removable storage components (e.g. magnetic tape, optical disk, flash memory drive, external hard drive, removable memory chip, etc.), network attached storage components (e.g. NAS drives), remote storage components (e.g. cloud-based storage) or the like.

The computing device (900) may include an external communications interface (930) for operation of the computing device (900) in a networked environment enabling transfer of data between multiple computing devices (900) and/or the Internet. Data transferred via the external communications interface (930) may be in the form of signals, which may be electronic, electromagnetic, optical, radio, or other types of signal. The external communications interface (930) may enable communication of data between the computing device (900) and other computing devices including servers and external storage facilities. Web services may be accessible by and/or from the computing device (900) via the communications interface (930).

The external communications interface (930) may be configured for connection to wireless communication channels (e.g., a cellular telephone network, wireless local area network (e.g. using Wi-Fi™), satellite-phone network, Satellite Internet Network, etc.) and may include an associated wireless transfer element, such as an antenna and associated circuitry.

The computer-readable media in the form of the various memory components may provide storage of computer-executable instructions, data structures, program modules, software units and other data. A computer program product may be provided by a computer-readable medium having stored computer-readable program code executable by the central processor (910). A computer program product may be provided by a non-transient computer-readable medium, or may be provided via a signal or other transient means via the communications interface (930).

Interconnection via the communication infrastructure (905) allows the one or more processors (910) to communicate with each subsystem or component and to control the execution of instructions from the memory components, as well as the exchange of information between subsystems or components. Peripherals (such as printers, scanners, cameras, or the like) and input/output (I/O) devices (such as a mouse, touchpad, keyboard, microphone, touch-sensitive display, input buttons, speakers and the like) may couple to or be integrally formed with the computing device (900) either directly or via an I/O controller (935). One or more displays (945) (which may be touch-sensitive displays) may be coupled to or integrally formed with the computing device (900) via a display (945) or video adapter (940).

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

Any of the steps, operations, components or processes described herein may be performed or implemented with one or more hardware or software units, alone or in combination with other devices. In one embodiment, a software unit is implemented with a computer program product comprising a non-transient computer-readable medium containing computer program code, which can be executed by a processor for performing any or all of the steps, operations, or processes described. Software units or functions described in this application may be implemented as computer program code using any suitable computer language such as, for example, Java™, C++, or Perl™ using, for example, conventional or object-oriented techniques. The computer program code may be stored as a series of instructions, or commands on a non-transitory computer-readable medium, such as a random access memory (RAM), a read-only memory (ROM), a magnetic medium such as a hard-drive, or an optical medium such as a CD-ROM. Any such computer-readable medium may also reside on or within a single computational apparatus, and may be present on or within different computational apparatuses within a system or network.

Flowchart illustrations and block diagrams of methods, systems, and computer program products according to embodiments are used herein. Each block of the flowchart illustrations and/or block diagrams, and combinations of blocks in the flowchart illustrations and/or block diagrams, may provide functions which may be implemented by computer readable program instructions. In some alternative implementations, the functions identified by the blocks may take place in a different order to that shown in the flowchart illustrations.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Throughout the specification and claims unless the contents requires otherwise the word ‘comprise’ or variations such as ‘comprises’ or ‘comprising’ will be understood to imply the inclusion of a stated integer or group of integers but not the exclusion of any other integer or group of integers.

1. An imaging assembly for an automated multimodal imaging system for imaging biological tissue comprising:
a support for supporting the tissue during imaging;
an optical 3D scanner for obtaining a plurality of 3D coordinates of a surface of the tissue;
an X-ray generator for sequentially radiating X-rays at a plurality of angles, through the tissue, toward an X-ray detector positioned below the tissue; and
an articulated arm arranged to hold an ultrasound transducer at an end thereof and to automatically move the ultrasound transducer along a path defined by the obtained 3D coordinates for ultrasound imaging of the tissue while maintaining the transducer in contact with the surface at a predetermined orientation.

2. The imaging assembly as claimed in claim 1 wherein the 3D scanner includes a camera positioned above the support and arranged to be moved relative to the support to sequentially position the camera at a plurality of angles relative to the support.

3. The imaging assembly as claimed in claim 1 wherein the 3D scanner includes a plurality of cameras positioned above the support and spaced apart from one another at a plurality of angles relative to the support.

4. The imaging assembly as claimed claim 1, wherein the X-ray generator is arranged to be moved relative to the support to sequentially position the X-ray generator at a plurality of angles relative to the support.

5. The imaging assembly as claimed in claim 1, further including a plurality of X-ray generators that are spaced apart from one another at a plurality of angles relative to the support.

6. The imaging assembly as claimed in claim 1, wherein the X-ray detector is a flat panel X-ray detector and is movable in a plane parallel to the support.

7. The imaging assembly as claimed in claim 1, wherein the predetermined orientation of the ultrasound transducer is in a direction substantially orthogonal to the surface of the tissue and wherein the articulated arm and/or ultrasound transducer includes a pressure sensor to measure a pressure between the ultrasound transducer and the surface of the tissue when in contact therewith.

8. An automated multimodal imaging system for imaging biological tissue, the system including an imaging computing device comprising:
a 3D coordinate obtaining module for receiving optical data from an optical 3D scanner and obtaining a plurality of 3D coordinates of a surface of the tissue from the received data;
an X-ray generator module configured to sequentially cause an X-ray generator to radiate X-rays at a plurality of angles, through the tissue, toward an X-ray detector positioned below the tissue;
an X-ray detector module for converting a sequence of X-rays received by the X-ray detector into X-ray data; and
an articulated arm module for automatically moving an articulated arm with an ultrasound transducer at an end thereof along a path defined by the obtained 3D coordinates, and for maintaining the transducer in contact with the surface of the tissue at an orientation for imaging of the tissue in a direction substantially orthogonal to the surface; and
an ultrasound module for obtaining ultrasound data from the ultrasound transducer.

9. The imaging system as claimed in claim 8 wherein the imaging computing device further includes an X-ray image module arranged to generate a plurality of two-dimensional X-ray images of the tissue from the X-ray data and an ultrasound image module for generating a plurality of two-dimensional ultrasound images of the tissue from the ultrasound data.

10. The imaging system as claimed in claim 8 wherein the 3D coordinate obtaining module is arranged to receive a plurality of digital images from one or more cameras, the digital images captured at a plurality of angles relative to the tissue and is further arranged to configure an angle of the one or more cameras relative to the tissue.

11. The imaging system as claimed in claim 8 wherein the X-ray generator controller is furthermore arranged to configure an angle of the X-ray generator relative to the tissue and the X-ray detector module is furthermore arranged to move the X-ray detector in a substantially horizontal plane.

12. The imaging system as claimed in claim 8 wherein the imaging computing device further includes a volumetric reconstruction module for generating, from the two-dimensional X-ray images, a first volumetric reconstruction of the tissue and for generating, from the two-dimensional ultrasound images, a second volumetric reconstruction of the tissue and an image co-registering module for co-registering the first and second volumetric reconstructions to generate a third volumetric reconstruction of the tissue.

13. The imaging system as claimed in claim 12 wherein the computing device further includes a slice reconstruction module for reconstructing image slices of one or more of the volumetric reconstructions, preferably by making use of tomosynthesis algorithms to reconstruct the image slices.

14. A method for automatically imaging biological tissue positioned on a support, the method comprising
obtaining a plurality of 3D coordinates of a surface of the tissue by means of a 3D scanner;
causing an X-ray generator to sequentially radiate X-rays at a plurality of angles, through the tissue, toward an X-ray detector positioned below the tissue;
converting a sequence of X-rays received by the X-ray detector into X-ray data; and
automatically moving an articulated arm having an ultrasound transducer at an end thereof along a path defined by the obtained 3D coordinates while maintaining the transducer in contact with the surface of the tissue at a predetermined orientation, and obtaining ultrasound data from the ultrasound transducer.

15. The method as claimed in claim 14 further including:
generating a plurality of two-dimensional X-ray images of the tissue from the X-ray data; and
generating a plurality of two-dimensional ultrasound images of the tissue from the ultrasound data.

16. The method as claimed in claim 15 wherein the biological tissue is breast tissue of a patient and for the step of providing the tissue to be imaged on the support to include:
donning the patient with a tight-fitting camisole made from material that is rendered acoustically transparent when impregnated with ultrasound gel;
impregnating the camisole over the breast tissue with ultrasound gel; and
positioning the patient in a supine position on the support.

17. The method as claimed in claim 16 wherein the 3D coordinates include 3D coordinates of both breasts of the patient and wherein the step of causing an X-ray generator to sequentially radiate X-rays at a plurality of angles through the tissue to include:
moving the X-ray detector into a first position, determined from the 3D coordinates, for detecting X-rays radiated through a first breast of the patient;
causing the X-ray generator to sequentially radiate X-rays at a plurality of angles, through the first breast, toward the X-ray detector positioned below the first breast;
moving the X-ray detector into a second position, determined from the 3D coordinates, for detecting X-rays radiated through a second breast of the patient; and
causing the X-ray generator to sequentially radiate X-rays at a plurality of angles, through the second breast, toward the X-ray detector positioned below the second breast

18. The method as claimed in claim 17 wherein the step of automatically moving the articulated arm and the ultrasound transducer along the path defined by the obtained 3D coordinates to be performed for both breasts using the same set of obtained 3D coordinates, such that the step of generating a plurality of two-dimensional ultrasound images of the tissue generates a plurality of two-dimensional images for both breasts of the patient.

19. The method as claimed in claim 16 wherein the two-dimensional X-ray images are X-ray images in a coronal plane and for the ultrasound images are ultrasound images in a sagittal plane.

20. The method as claimed in claim 15 further including the steps of:
generating, from the X-ray images, a first volumetric reconstruction of the tissue;
generating, from the ultrasound images, a second volumetric reconstruction of the tissue; and
co-registering the first and second volumetric reconstructions to generate a third volumetric reconstruction of the tissue.
